(19) United States
(12) Patent Application Publication
(10) Pub. No.: US 2003/0078549 A1
(43) Pub. Date: Apr. 24, 2003

(54) Microfabricated surgical devices and methods of making the same

(76) Inventors: Philip Anthony Stupar, Oxnard, CA (US); Albert P. Pisano, Danville, CA (US)

Correspondence Address: FISH & RICHARDSON P.C., 500 ARGUELLO STREET, SUITE 500, REDWOOD CITY, CA, 94063, US

(21) Appl. No.: 10/165,645

(22) Filed: Jun. 6, 2002

Related U.S. Application Data
(60) Provisional application No. 60/297,020, filed on Jun. 8, 2001.

(51) Int. Cl. A61M 5/32

(52) U.S. Cl. 604/272

(57) ABSTRACT

This invention relates to microfabricated surgical devices and methods of making the same. One such device includes an end portion and a body portion wherein at least a part of the body portion is hollow and includes a conformally coated polymer formed on inside and outside surfaces of the body portion. One such method includes defining at least one channel in the surface of a first substrate, joining a second substrate to the first substrate to cover the channel, forming a trench in the first and second substrates on each side of the channel to define a shell structure, and releasing the shell structure from the first and second substrates.

### RELATED APPLICATIONS

This application claims the benefit of U.S. Provisional Application No. 60/297,020 filed Jun. 8, 2001, which is incorporated herein by reference.

### TECHNICAL FIELD

This invention relates generally to surgical devices, and more particularly to microfabricated surgical devices and methods of making the same.

### BACKGROUND

With the development of micro-fluidic systems on a chip comes the need for these chips to interact with the outside world. Microfabricated surgical devices, such as microneedles, are one such way to introduce samples to and extract solutions from organic tissue. However, current silicon and polysilicon microneedles fracture easily, and therefore must have their strength and toughness increased in order to be truly effective fluidic interconnects.

Out-of-plane, single crystal silicon microneedles can be made very sharp, but are limited in length by the thickness of the wafer from which they are made, and are somewhat fragile because the tips must be made hollow to facilitate fluid transport. In-plane single crystal silicon needles use deposited films to cap the fluid channel, and therefore have thin top wall thicknesses that can fracture under bending loads. Polysilicon microneedles use a deposited film for the entire structural layer and therefore are also likely to fracture under relatively small loads.

Although such previously fabricated microneedles have been proven to be effective fluidic interconnects, they have not been integrated into commercial devices because of the lack of strength and toughness. In addition, their brittle nature makes them hazardous to patients.

Such silicon microneedles, for instance, will fracture before undergoing any plastic deformation. Such failure can be catastrophic. This type of failure is particularly hazardous for a microneedle application because this sort of rupture can lead to leakage of chemicals into the body that can be lethal in large dosages. Additionally, leaving behind particles of silicon in the body can have very perilous effects.

### SUMMARY

In one aspect, an embodiment of the invention features a microfabricated surgical device comprising an end portion and a body portion wherein at least a part of the body portion is hollow and includes a conformally coated polymer formed on inside and outside surfaces of the body portion.

Various implementations of the invention may include one or more of the following features. The polymer is Parylene, and the end portion and the body portion are silicon. The Parylene is deposited by gas vapor deposition. The polymer is selected from the group consisting of Parylene N, Parylene C, Parylene D, polystyrene or Teflon®. A catheter is joined to the device opposite the end portion. An interior cross-sectional dimension of the body portion is between about 25 and 200 microns. An exterior cross-sectional dimension of the body portion is between about 50 and 700 microns. The microfabricated device has a length of between about 1 and 10 millimeters.

In another aspect, an embodiment of the invention features a microfabricated needle. The needle has a tip and a shaft wherein at least the shaft includes a hollow portion having a conformal polymer layer formed on an inside surface and an outside surface of the shaft.

Various implementations of the invention may include one or more of the following features. The end portion and the body portion are silicon, and the polymer is selected from the group consisting of Parylene N, Parylene C, Parylene D, polystyrene, or Teflon®. The microfabricated needle includes a fluid entry port and a fluid exit port. An end of the hollow portion is in fluid communication with the catheter. An interior cross-sectional dimension of the shaft is between about 25 to 200 microns, an exterior cross-sectional dimension of the shaft is between about 50 and 700 microns, and the microfabricated needle has a length of between about 1 and 10 millimeters. The tip of the microfabricated needle is either solid or hollow.

In still another aspect, an embodiment of the invention features a method of making a microfabricated surgical device. The method comprises: defining at least one channel in a surface of a first substrate, joining a second substrate to the first substrate to cover the channel, forming a trench in the first and second substrates on each side of the channel to define a shell structure, and releasing the shell structure from the first and second substrates.

Various implementations of the invention may include one or more of the following features. The channel is etched into the first substrate. The first substrate is joined to the second substrate by a fusion bonding process. The trench is located on each side of the channel by an infrared alignment technique. The first substrate is a silicon wafer and the second substrate is a silicon on insulator wafer. The shell structure is released by etching the insulator of the silicon on insulator wafer. The plurality of channels are defined in the surface of the first substrate to form a plurality of shell structures.

In yet another aspect, an embodiment of the invention features a method of making a microfabricated surgical device. The method comprises: defining a channel in the surface of a first substrate, joining a second substrate to the first substrate to cover the channel, forming a trench in the first and second substrates on each side of the channel to define a shell structure, releasing the shell structure having a hollow portion from the first and second substrates, and conformally depositing a polymer on the inside and outside surfaces of the shell structure.

Various implementations of the invention may include one or more of the following features. The polymer is Parylene. The polymer is deposited by gas vapor deposition. The polymer is selected from the group consisting of Parylene N, Parylene C, Parylene D, polystyrene or Teflon®.

An advantage of the invention is that it provides a microfabricated needle that can withstand large forces without fracturing. The microfabricated needles can have large wall thicknesses between about 35 micron (μm) and 100 μm. The needles, depending on their wall thickness, can withstand bending movements on the order of about 0.5 mNm and 1.56 mNm. They can also puncture very tough membranes having thicknesses on the order of 150 μm to 400 μm.

The strength and toughness of these needles provide greater yields in manufacturing, fewer failures in the field, and less expensive packaging solutions for shipment. The deposition of a conformal polymer layer provides a laminated structure that increases the toughness of the needle.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D are schematic, cross-sectional views illustrating steps in the fabrication of a microfabricated needle.

FIG. 2 is a schematic, perspective view illustrating microfabricated needles.

FIG. 3 is a schematic view illustrating a polymer and silicon laminated shell of a microfabricated needle structure.

Like reference symbols and reference numbers in the various drawings indicate like elements.

### DETAILED DESCRIPTION

The present invention is directed to microfabricated surgical devices and methods of making the same. The present invention will be described in terms of several representative embodiments and processes in fabricating a microfabricated needle or microneedle. The described processes may also be used to make other microfabricated surgical devices, such as neural probes, lancets, in-vivo biological assay systems, cutting microtools, or devices including microtubing and incorporating, for example, channels and mixers.

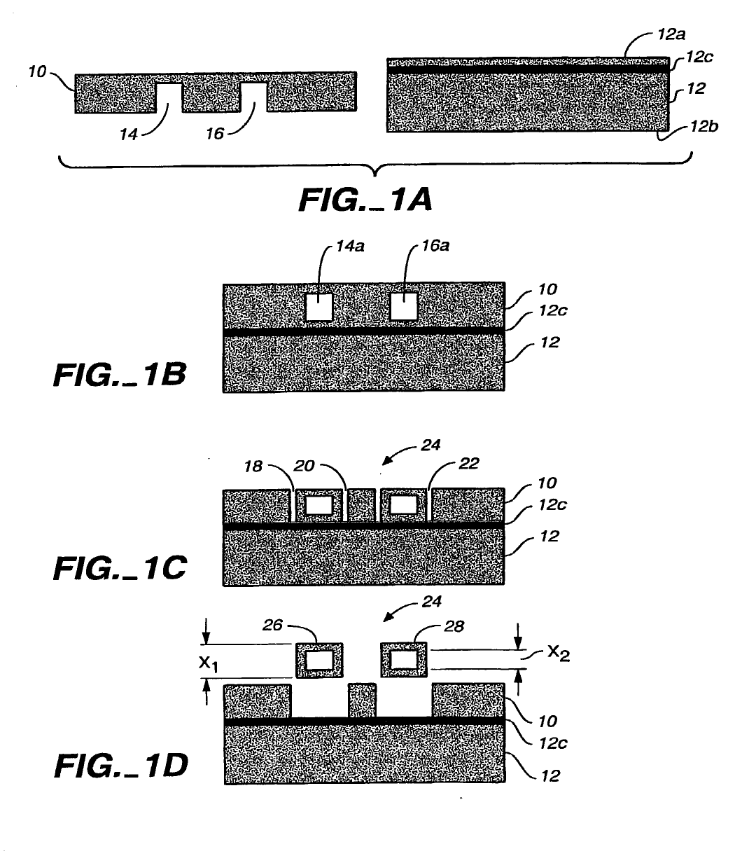

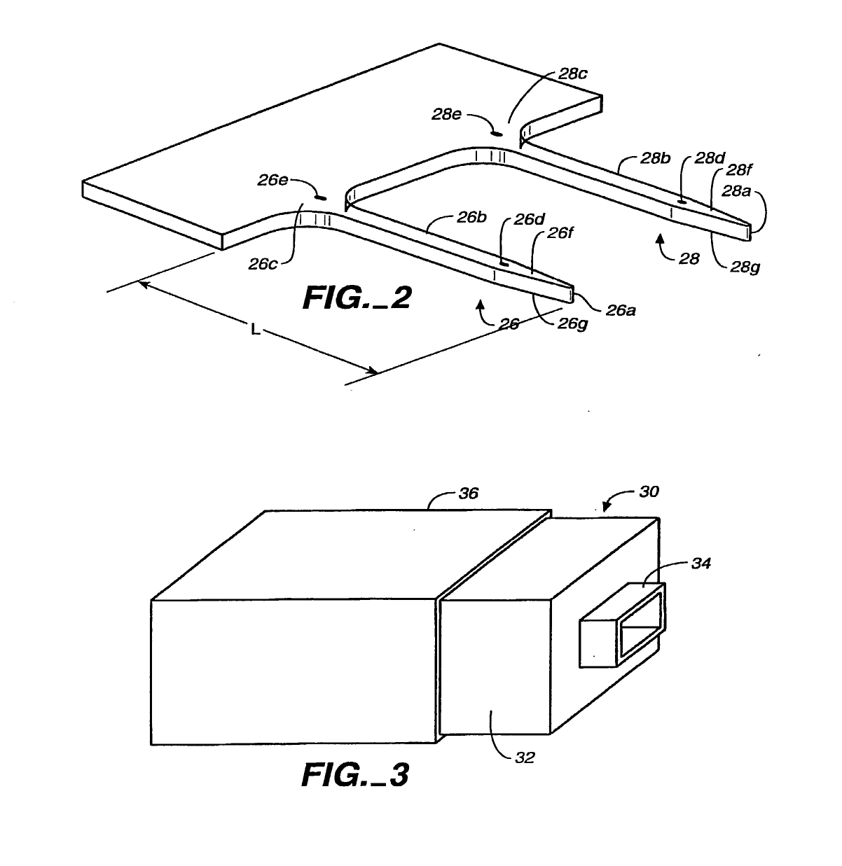

As shown in FIG. 1A, the fabrication of a microfabricated surgical device, such as a microfabricated needle or microneedle 26 or 28 (see FIG. 2), may start with two substrates or wafers such as a <100> single crystal silicon wafer 10 and a Silicon on Insulator (SOI) wafer 12. The wafer 10 is typically around 200 to 500 microns (μm) thick. More typically, the wafer 10 is about 200 μm thick. The thickness of the wafer 10 will define the overall thickness of the device. This wafer 10 is patterned using, for example, photoresist (PR) lithography, to define where channels 14 and 16 are to be formed. The wafer 10 is then etched, for example, in an STS deep silicon etcher, to form the channels 14 and 16. Other etch techniques, such as wet, dry, anisotropic or isotropic etching, could also be used.

The etch depth, and in turn the remaining wafer thickness, will define the top wall thickness of the shell. The channels outline the needle structure, and they can have vertical sidewalls.

The wafer 12 may be between about 500 and 700 μm thick. The wafer 12 includes a first layer of silicon 12a joined to another layer of silicon 12b by a silicon dioxide layer 12c. The thickness of the wafer layer 12a will define the bottom wall thickness of the shell.

The substrates 10 and 12 could be other materials. For example, the wafer 12 could be a glass wafer epoxy bonded to a handle.

The wafers 10 and 12 are fusion bonded together to form buried channels 14a and 16a that correspond to channels 14 and 16, respectively (FIG. 1B). This bond may be performed in two steps. First a pre-bond is performed in which the two clean wafers 10 and 12 are brought into close proximity allowing Van Der Wall forces to temporarily hold the wafers together. This pre-bond is performed with two clear hydrophobic bare silicon surfaces. This is important because even a thin native oxide layer could be etched away during the release, therefore separating the two wafers. It is also imperative to perform the pre-bond immediately following a spin rinse-dry. Wafers that are not particle free will have small voids that will lead to incomplete bonding of the shell structure. The pre-bonded wafers are then annealed at 1000° Centigrade (C) for one hour to allow the diffusion between the two wafers to permanently bond them together.

Alternatively, the wafers may be adhered together by curing of thermoset photoresists. Also, the wafers may be bonded by such techniques as anodic, metal compression or epoxy/photoresist bonding.

As shown in FIG. 1C, the bonded wafers 10 and 12 are then patterned with trenches, for instance trenches 18, 20 and 22 that define the shape of a shell structure 24. PR lithography may be used to pattern these trenches, and the trenches may have vertical sidewalls. The depth of the trenches 18, 20 and 22 may be between about 50 and 700 μm, and more typically between about 50 and 300 μm.

The trenches 18, 20 and 22 are aligned to the buried channels 14a and 16a using, for example, infrared (IR) alignment techniques in which IR light is used to look through the wafer. The buried channels show up as shadows which can be aligned to with an accuracy of approximately 3 μm. This pattern is etched through the bonded wafers down to the buried silicon dioxide layer 12c of the wafer 12 using, for example, a STS silicon etcher.

If other alignment techniques are used, the alignment to the buried channel can be improved. For instance, the buried channels can be aligned with an accuracy of about 0.5 μm, if a front to back alignment mask transfer technique is used.

The oxide layer 12c is then etched using concentrated hydrofluoric acid (HF) and the structure 24 is released from the wafer (FIG. 1D). The structure 24, in this case, consists of two needles 26 and 28 (see also FIG. 2).

Alternatively, the structure may consist of one or more than two such needles. For instance, if a single needle is to be made only one channel would be needed in wafer 10 and trenches would be formed on each side of the channel. On the other hand, several thousand needles can be fabricated, for example, on a four-inch diameter wafer, leading to device batch fabrication.

This fusion bonded shell process can be used to fabricate micro-needles for fluidic interconnects between micro-fluidic devices and the outside world. As shown in FIG. 2, the microneedles 26, 28 generally have a body portion and an end portion. More specifically, the microneedles include a needle tip 26a, 28a; a needle shaft 26b, 28b; and a needle base 26c, 28c. The needle tip or termination point 26a, 28a provides a penetration edge wherein a top surface 26f, 28f of the needle tip is a projection of its bottom surface 26g, 28g. A needle can also be made such that its tip forms an insertion or penetration point. The insertion point is advantageous as less force is necessary to break tissue than with an insertion edge micro-needle. Such a needle tip is described in application Ser. No. 09/877,653, filed Jun. 8, 2001, entitled Microfabricated Surgical Device assigned to the assignee of the subject application, the entire disclosure of which is incorporated herein by reference.

Each needle also includes ports 26d, 28d and ports 26e, 28e. The ports 26d, 28d are etched into the end of the needles, and the ports 26e, 28e are etched in the bases of the needles. An outlet port may be unnecessary if a fluid is taken into the base of the needle acting as a micro-fluidic chip.

The ports may be formed by deep reactive ion etching (DRIE). The ports could also be etched using other silicon etching techniques.

The needle shaft and a channel in the needle base are hollow, permitting the withdrawal of a fluid, for instance, from a patient via the needle ports. In such a configuration, the needle ports 26d, 28d function as inlet or entry ports, while the ports 26e, 28e function as outlet or exit ports. If a fluid, for instance, was to be injected into a patient then the ports 26d, 28d would operate as the outlet ports, while the ports 26e, 28e would function as the inlet ports.

Single crystal silicon fusion bonded needles have very sharp tips. Because the tip sharpness is defined by lithography and a silicon etch, there is essentially no tip rounding, and therefore, very tip sharpness can be achieved.

Strength is one of the top concerns in the fabrication of micro-needles. One advantage of the silicon fusion bonded shell process is that each of the shell wall thicknesses are independently controlled and have a very large range of possible dimensions. The bottom wall thickness is defined by the thickness of the device layer in the original SOI wafer 12. This thickness can be as small as a micron and as large as a full wafer thickness, around 500 μm. The top wall thickness is defined by the depth of the fluid channel etch 14, 16 and the original thickness of the wafer 10. Theoretically, this thickness could be as small as a few microns. In addition, if yield is not a concern, smaller thicknesses can be achieved by allowing the etch to go through the wafer in some sections. The maximum thickness of the top wall is only limited by the original wafer 10 thickness, around 500 μm. The side wall thicknesses are defined solely by lithography and can therefore range from a few microns to the size of the chip, around 1 cm.

By way of example, as shown in FIGS. 1D and 2, the length L of these needles range from about 1 to 10 millimeters (mm), and more typically between about 4 and 6 mm. The exterior cross-sectional dimension x₁ of the needle shaft may be between about 50 and 700 μm, and more typically between about 50 and 300 μm. The hollow interior cross-sectioned dimension x₂ of the needle shaft may be on the order of 25 to 200 μm, and more typically between about 40 and 100 μm.

The complete control over the shell dimensions allows for unique needle designs. Single crystal silicon fusion bonded shells can be fabricated with completely solid bases by only extending a fluid channel (not shown) to the outlet port. This solid base is very robust and allows for easy integration and needle handling. The base 26c, 28c, for instance, can be a large area that provides a mechanism for handling or assembly of the micro-needles. The base, however, may be eliminated, if, for instance, a needle is to be placed at the tip of a catheter for use in interventional procedures. For example, a catheter tip can be lined up with a needle shaft end and as a polymer grows to create a laminated needle structure, as discussed below, it encapsulates the catheter tip, fixing the needle in place.

Single crystal silicon fusion bonded micro-needles can have completely solid tips as well. Through the use of an inlet port etched into the top face sheet of the needle, the fluid channel can end at the inlet port allowing for a stronger, solid silicon tip. The needle tip could also be hollow.

The micro-needle structure discussed above was formed with vertical sidewalls (see FIG. 1A). However, other sidewall geometries are possible, depending upon the etching technique used and the crystallographic microstructure of the single crystal silicon. Rounded features can be made in the plane of the wafer using isotropic wet chemical etching of silicon, and sloping sidewalls can be formed by anisotropic wet chemical etching. These sidewall geometries may be useful for different device configurations, for example, micro-needles with filter plates or surgical devices that can cut sideways. Also the fluid channels can be patterned with devices such as filters, pumps, valves or electrodes.

Because silicon is a brittle material and will fracture before undergoing any plastic deformation, failure is catastrophic. This type of failure is particularly hazardous for a micro-needle application because this type of rupture can lead to leakage of chemicals into the body that can be lethal in high dosages. In addition, leaving behind particles of silicon in the body can also have very perilous effects. Although most micro-needle designs should be strong enough to withstand the loads required to function properly, extra precautionary steps can be taken to insure the safety of the patient. To this end, as shown in FIG. 3, a polymer and silicon laminated micro-shell 30 can be used to form a needle.

To fabricate polymer and silicon laminated shells, the fusion bonded shell process is run first. However, before the wafer is diced into chips and the tethers are broken to release the needles, a conformal polymer deposition is performed. Specifically, a Parylene C polymer can be gas vapor deposited onto a shell structure 32. Parylene is the generic name for the polymer poly-para-xylylene. Parylene C is the same monomer modified by the substitution of a chlorine atom for one of the aromatic hydrogens. Parylene C was chosen because of its conformality during deposition and its relatively high deposition rate, around 5 μm per hour.

The Parylene process is a conformal vapor deposition in which the substrate is kept at room temperature. A solid dimmer is first vaporized at 150° C. and then cleaved into a monomer at 650° C. This vaporized monomer is then brought into the room temperature deposition chamber where it absorbs and polymerizes onto the substrate. Because the mean free path of the monomer gas molecules is on the order of 0.1 cm, the Parylene deposition is very conformal. The Parylene coating is pin hole free at below a 25 nanometer (nm) thickness.

Due to the extreme conformality of the deposition process, Parylene coatings 34 and 36 will coat the inside and outside of the hollow portion of the shell 32, respectively, to form a Parylene/silicon/Parylene laminated structure. The Parylene coating will not only protect the outside of the silicon shell from fracture and separation from the device, the coating on the inside of the shell will stop the leakage of any fluids being transported in the event of the fracture of the silicon section.

The Parylene coating 34 inside the shell and the Parylene coating 36 outside the shell may be on the order of 0.5 to 30 μm thick, and more typically about 5 μm thick.

Other Parylenes, such as Types N and D, may be used in place of Parylene C. Also, other polymers, such as Teflon® or polystyrene, can be used. The important thing is that the polymer be conformally deposited. That is, the deposited polymer has a substantially constant thickness regardless of surface topologies or geometries.

Additionally, a fluid flood and air purge process could be used to form a conformal polymer layer in and outside the shell. Polymers that may be used in this process include polyurethane, an epoxy or a photoresist.

The silicon fusion bonded shell process was designed to fabricate shells with relatively large wall thicknesses that could withstand the sizeable forces necessary for a structure to interact with the outside world. These shells are particularly suited to the application of micro-needles. These stronger shells can withstand the forces required to puncture touch membranes.

Relatively large axial forces are required to puncture a membrane with a silicon micro-needle. This type of compressive axial force can lead to the failure of a micro-needle by Euler buckling. Buckling occurs when there is an instability due to the restoring force for an infinitesimal deformation being lower than the moment caused by the deformation. Under the assumption of Euler buckling for a column, the maximum compressive load that a structure can support in compression is given by:

$${F}_{cr} = \frac{{π}^{2} EI}{{CL}^{2}} \qquad ( Eq . 1 )$$

with a Young's Modulus E, a length L, an end condition factor C, and an area moment of inertia I given by:

$$I = \frac{{b}_{o} {h}_{o}^{3} - {b}_{i} {h}_{i}^{3}}{12} \qquad ( Eq . 2 )$$

where b is the inside and outside width and h is the inside and outside height of the shell structure. The end condition factor C is determined by the loading conditions. Under the assumptions that the needle base is fixed to a large structure, and the needle tip is simply supported by the membrane to be punctured, the end condition factor is 0.49. For typical silicon fusion bonded dimensions, length of 4.5 mm, outside width and height of 200 μm, and inside width and height of 100 μm, the maximum endurable compressive load is 19.9 N.

In order to determine if the Euler buckling assumption is accurate, the slenderness ratio (L/k) must be compared to the critical slenderness ratio (L/k)_{cr}. The Euler buckling assumption is valid if the slenderness ratio of the needle is larger than the critical value. Using the definition of the slenderness ratio, this gives an Euler buckling (Eq. 3) tion of:

$$( \frac{L}{k} ) = \sqrt{\frac{LA}{I}} > {( \frac{L}{k} )}_{cr} = \sqrt{\frac{{π}^{2} E}{2 {σ}_{y}}}$$

Using the dimensions for the silicon micro-needles, the slenderness ratio is around 850, which is much smaller than the critical slenderness ratio, around 11. This verifies that the Euler buckling assumption is valid.

A needle will fail in bending when the stress caused by the bending moment, given by:

$$σ = \frac{FLc}{I} \qquad ( Eq . 4 )$$

exceeds the fracture strength of the material. This gives a maximum endurable bending load of:

$$F = \frac{I {σ}_{fr}}{Lc} \qquad ( Eq . 5 )$$

Using the typical dimensions discussed immediately above with a fracture strength of silicon, σ_{fr} taken as 7 Gpa, the critical bending force is 1.9 N.

Although the critical bending load is lower than the critical Euler buckling load, it is not safe to say that the critical failure mode will be bending stress. The compressive force endured by the needle during the penetration of a membrane could also be much higher than the bending forces endured by movement of the needle. The silicon fusion bonded needles must therefore be designed so that each of the forces is kept below the critical values.

In order to verify the usefulness and strength of silicon fusion bonded needles, their stiffness, puncture loads, and maximum withstandable bending moments were measured. In addition, needle insertion, retraction, and fluid extraction were performed with these needles.

To prove the validity of silicon micro-needles, puncture tests were performed. Single crystal silicon fusion bonded needles were able to pierce a wide range of materials including raw lamb meat, chicken breasts with and without skin, 150 μm thick rubber membranes, and 400 μm gelatin membranes.

The insertion force for a silicon micro-needle into a gelatin membrane was measured using a force transducer attached to a slider and fine adjust screw. The slider constrains the motion of the micro-needle to only vertical deflections. The fine adjust screw was used to lower the micro-needle into the membrane at a very slow, constant decent. The insertion force was found to linearly increase as the needle deflected the gelatin membrane. Then, the force drops off dramatically as the needle tip pierces the membrane. However, as the tapered section of the needle penetrates the membrane and opens up the hole, the insertion force once again increases. Once the tapered section has been completely inserted past the membrane, the force once again drops off and reaches a nominal value of the friction force on the needle.

The maximum load on the silicon micro-needle, in one case, during the piercing of the gelatin membrane was 0.45 N. This value is well below the critical Euler buckling load of 19.9 N calculated above. Therefore, it is safe to say that Euler buckling is not the critical failure mode for these needles, and therefore their strength should be determined by the maximum bending load that they can endure.

The silicon fusion bonded micro-needles were not only able to pierce a gelatin membrane, but were also able to extract fluid from within a gelatin capsule. This fluid was extracted using the internal pressure of the gelatin capsule to pump the fluid into the inlet port at the tip of the needle, through the needle channel, and out the exit port.

The stiffness and strength of the single crystal silicon fusion bonded micro-needles were also tested. Using at least squares linear fit through the origin, the measured bending stiffness was 680 N/m. The total error in the stiffness measurement for the range of forces and displacements in this experiment was 1.6%.

The theoretical bending stiffness is given by:

$$k = \frac{F}{x} = \frac{3 EI}{{L}^{1}} \qquad ( Eq . 6 )$$

E is the Young's Modulus of single crystal silicon (160 Gpa), L₁ the length to the loading point (4.46 mm), and I is the area moment of inertia given by:

$$I = \frac{{b}_{o} {h}_{o}^{3}}{12} - \frac{{b}_{i} {h}_{i}^{3}}{12} \qquad ( Eq . 7 )$$

where bₒ and hₒ are the width and height of the overall shell (both 200 μm), and b₁ and hᵢ are the width and height of the inside channel (both 100 μm). Using these equations, the theoretical bending stiffness for the tested needle was 675 N/m. The error of the theoretical stiffness versus the measured value was 0.8%. This error is well within the experimental error of 1.6%.

The fracture strength of the single crystal silicon fusion bonded micro-needles was determined by measuring the maximum bending moment sustainable by a specimen. In this one experiment, a load was slowly applied to cantilevered micro-needles until fracture occurred. The bending moment was automatically measured in 0.5 second intervals by a load cell and digital multimeter attached to a personal computer. The bending moment increased over time until fracture occurred, causing the load to quickly return to zero.

The maximum bending moment was measured for micro-needles with varying wall thicknesses. These measurements were performed multiple times for each specimen size and the average bending moments sustained for each needle design is shown in Table 1.

TABLE 1

| | Molded Polysilicon | Single Crystal Silicon | Single Crystal Silicon |
|---|---|---|---|
| Needle Specimen | (20 μm waIls) | (37 μm walls) | (50 μm walls) |
| Ave. Max. Moment | 0.25 mNm | 0.54 mNm | 1.56 mNm |

As shown in Table 1, on average, the thick walled silicon micro-needles sustained over six times the bending moment of a polysilicon micro-needle.

The parylene and silicon laminated needles were designed to have the strength of the silicon fusion bonded needles, and the toughness that is usually associated with polymers. The addition of the parylene layers has no effect on the stiffness or maximum bending moment sustained by the needles. However, to test the increase in toughness, the parylene and silicon laminated needles were tested for maximum bending deflection, fluid extraction through fractured needles, and fractured needle extraction from a pierced membrane. All the needles tested had an outside and inside Parylene layer that was 5 μm thick and a silicon layer that was 37.5 μm thick.

Although the silicon fusion bonded needles are extremely strong, they are brittle and can therefore fracture without warning. However, the parylene and silicon laminated needles can fracture and undergo large plastic deformations without failing. The Parylene layer is tough enough to hold the needle together during the fracture of the silicon layer. To test how tough these laminated shells were, the maximum bending rotation for a needle with a fractured silicon layer was tested. These laminated needles withstood very large rotations without failing. In addition, the outside Parylene layer stayed completely intact during large rotations. In fact, the Parylene and silicon laminated shells underwent complete 180° rotations without detaching from the base. In addition, although the needles went through up to 20 complete 180° reversals, the Parylene layer never failed due to fatigue during the course of the experiments.

The Parylene and silicon laminated needles have been shown to withstand multiple, very large deflections without detaching from the needle base. In addition, to show that these needles with fractured silicon layers can still function, fluid extraction experiments were performed with the laminated needles bent at angles up to 45°. The bent needles were still able to extract fluids from a pierced membrane without leaking. Even though some specimens had ruptured outer Parylene layers, the inner Parylene layers were able to maintain the integrity of the fluid channel and transport the fluid out of the needle exit port. This shows that even if a needle fractures after it has been injected into a body, the needle will not leak and can even continue to function by extracting or delivering fluids.

A big concern of the use of a brittle material in the fabrication of needles is the fear of leaving behind parts of the needle inside the pierced body. To show that a Parylene and silicon laminated needle is safe in these respects, needle extraction experiments with fractured needles were performed. In these experiments, laminated needles with fractured silicon layers were extracted from pierced membranes. These experiments were performed with needles with two fractures, one inside and one outside the pierced body. A needle with a fracture both inside and outside the pierced membrane can be completely removed without leaving behind needle parts.

The silicon fusion bonded shell process is ideal for fabricating shells with wall thicknesses large enough to withstand the forces of the outside world. This process can be used for fluidic interconnects such as micro-needles that must puncture tough membranes, and therefore must be able to withstand large forces without breaking. Because all of the shell wall thickness in the silicon fusion bonded shell process are defined by either lithography or wafer thicknesses, they can be fabricated as large or small as needed for their specific application. Silicon fusion bonded needles have been proven to withstand very large forces.

Although silicon fusion bonded needles are strong enough to be used as hypodermic injection needles, they are still safety risks because they can fail. To improve the toughness of silicon fusion bonded needles, Parylene coatings, as noted, can be deposited onto the needles to form Parylene and silicon laminated shells. These needles have the strength of the silicon fusion bonded structures with a much increased toughness. These laminated needles remain intact and functioning even when the silicon layer fractures. Therefore, the Parylene and silicon laminated needles are strong enough to be used as hypodermic injection needles, and are tough enough to be used without worrying about a catastrophic failure that could put the patient's safety at risk.

Microfabricated needles can be used to inject pharmaceutical agents into or extract biological samples from humans or animals while limiting injury or pain. The scale of these microneedles allows insertion into the human epidermis without penetrating deep enough for nerve reception. One application of this technology is insulin injection for diabetics who need a daily dosage of medication where pain and possible scarring occur with each conventional needle penetration.

These devices can also be used for interventional surgical methods in which a microneedle attached to the distal (inside the body) end of a catheter could penetrate an arterial wall with a microscale hole. Medical research has shown that damage to the inside of arteries caused by abrasion or lesion can seriously affect patients with sometimes drastic consequences such as vasospasm, leading to arterial collapse and loss of blood flow. Breach of the arterial wall through interventional surgical microneedles can prevent such problems.

The use of interventional surgical microneedles also allows highly localized pharmaceutical injections without the limitation of remaining external to the body. Common pharmaceutical procedures carried out with intravascular injections cause unnecessary flushing of the drugs throughout the body and filtering through the kidneys liver and the lymphatic system. On the other hand, localized injections allow slow, thorough integration of the drug into the tissue, thus performing the task more efficiently and effectively, saving time, money, drugs, and lives.

The microfabricated needle tip, for certain applications, can be coated with a blood-clotting agent such as heperin. These microneedles can also be used to introduce fluids to and extract fluids from a micro-fluidic system on a chip.

A number of embodiments of the invention have been described. Nevertheless, it will be understood that various modifications may be made without departing from the spirit and scope of the invention. Accordingly, other embodiments are within the scope of the following claims.

We claim:

1. A microfabricated surgical device comprising:
an end portion and a body portion wherein at least a part of the body portion is hollow and includes a conformally coated polymer formed on inside and outside surfaces of the body portion.

2. The microfabricated device of claim 1 wherein the polymer is Parylene, and the end portion and the body portion are silicon.

3. The microfabricated device of claim 2 wherein the Parylenee is deposited by gas vapor deposition.

4. The microfabricated device of claim 1 wherein the polymer is selected from the group consisting of Parylene N, Parylene C, Parylene D, polystyrene, or Teflon®.

5. The microfabricated device of claim 1 wherein a catheter is joined to the device opposite the end portion.

6. The microfabricated device of claim 1 wherein an interior cross-sectional dimension of the body portion is between about 25 and 200 microns.

7. The microfabricated device of claim 1 wherein an exterior cross-sectional dimension of the body portion is between about 50 and 700 microns.

8. The microfabricated device of claim 1 having a length of between about 1 and 10 millimeters.

9. A microfabricated needle comprising:
a tip and a shaft wherein at least the shaft includes a hollow portion having a conformal polymer layer formed on an inside surface and an outside surface of the shaft.

10. The microfabricated needle of claim 9 wherein the end portion and the body portion are silicon, and the polymer is selected from the group consisting of Parylene N, Parylene C, Parylene D, polystyrene, or Teflon®.

11. The microfabricated needle of claim 9 further including a fluid entry port and a fluid exit port.

12. The microfabricated needle of claim 11 wherein an end of the hollow portion is in fluid communication with a catheter.

13. The microfabricated needle of claim 9 wherein an interior cross-sectional dimension of the shaft is between about 25 to 200 microns, an exterior cross-sectional dimension of the shaft is between about 50 to 700 microns, and the microfabricated needle has a length of between about 1 and 10 millimeters.

14. The microfabricated needle of claim 9 wherein the tip is solid or hollow.

15. A method of making a microfabricated surgical device comprising:
defining at least one channel in a surface of a first substrate;
joining a second substrate to the first substrate to cover the channel;
forming a trench in the first and second substrates on each side of the channel to define a shell structure; and
releasing the shell structure from the first and second substrates.

16. The method of claim 15 wherein the channel is etched into the first substrate.

17. The method of claim 16 wherein the first substrate is joined to the second substrate by a fusion bonding process.

18. The method of claim 16 wherein the trench is located on each side of the channel by an infrared alignment technique.

19. The method of claim 16 wherein the first substrate is a silicon wafer and the second substrate is a silicon on insulator wafer.

20. The method of claim 19 wherein the shell structure is released by etching the insulator of the silicon on insulator wafer.

21. The method of claim 15 wherein a plurality of channels are defined in the surface of the first substrate to form a plurality of shell structures.

22. A method of making a microfabricated surgical device comprising:
defining a channel in a surface of a first substrate;
joining a second substrate to the first substrate to cover the channel;
forming a trench in the first and second substrates on each side of the channel to define a shell structure;
releasing the shell structure having a hollow portion from the first and second substrates; and
conformally depositing a polymer on inside and outside surfaces of the shell structure.

23. The method of claim 22 wherein the polymer is Parylene.

24. The method of claim 22 wherein the polymer is deposited by gas vapor deposition.

25. The method of claim 22 wherein the polymer is selected from the group consisting of Parylene N, Parylene C, Parylene D, polystyrene or Teflon®.
